(19) United States
(12) Patent Application Publication
(10) Pub. No.: US 2024/0409905 A1
(43) Pub. Date: Dec. 12, 2024

(54) Method of Harvesting Biologics

(71) Applicant: Solventum Intellectual Properties Company, Maplewood, MN (US)

(72) Inventors: Kenneth C. Koehler, Woodbury, MN (US); Alexei M. Voloshin, Woodbury, MN (US); Ki-Joo Sung, Minneapolis, MN (US); Narendranath Bhokisham, Saint Paul, MN (US); Andrew W. Vail, Bayport, MN (US); Kristopher E. Richardson, Inver Grove Heights, MN (US); Rebecca A. Hochstein, St. Paul, MN (US)

(21) Appl. No.: 18/689,462

(22) PCT Filed: Aug. 4, 2022
(86) PCT No.: PCT/IB2022/057281

§ 371 Date: Mar. 6, 2024

Related U.S. Application Data
(60) Provisional application No. 63/241,672, filed on Sep. 8, 2021.

(51) Int. Cl. C12N 7/00

(52) U.S. Cl. CPC C12N 7/00; C12N 2750/14121; C12N 2750/14152

(57) ABSTRACT

A method. The method includes providing a filter medium comprising a functionalized nonwoven; passing a first fluid containing cells through the filter medium, wherein at least some of the cells are captured by the functionalized nonwoven; passing a second fluid, and optionally a third and/or fourth fluid through the filter medium, wherein the second and/or third and/or fourth fluid disrupts at least one of the captured cells; and recovering an intracellular biological product in the first and/or second and/or third and/or fourth fluid.

### BACKGROUND

Biologics such as recombinant proteins and gene therapy vectors are often generated using cell culture techniques. Host cells (i.e. mammalian, insect, bacteria or other cell line) are harnessed to produce the therapeutic of interest. Chinese hamster ovary (CHO) cells are the most commonly used cell line in the industry based on their ability to adapt and grow in suspension, grow in serum-free chemically defined medium, high production capabilities, post translational modifications, and more. CHO cells account for >70% of produced protein therapeutics but these biologics can be produced in several systems including microbes, plants, insects, and/or other mammalian cells. Whole cells and cell debris are typically removed, resulting in a clarified cell culture fluid containing the desired biologic. The clarified cell culture product can then be subjected to additional purification steps to bolster purity and concentration.

### SUMMARY

In biopharmaceutical manufacturing, there is a need to separate the target biomolecule of interest, such as intracellularly expressed biomolecules, from a feedstock comprising cells. When a biomolecule of interest is expressed intracellularly, a cell disruption step is often required to release the desired product. Cells are generally suspended in a cell culture fluid or placed in another buffer system, such as a lysis buffer, when a lytic step is required for recovery of an intracellularly expressed target biopharmaceutical molecule. The resulting lysate then requires additional purification to isolate the desired pharmaceutical from the cell debris and other contaminants released during the lysis procedure. Therefore, a single use primary clarification step that can replace centrifuges, tangential flow microfiltration, and conventional depth filters as the primary clarification step is needed.

Thus, in one aspect, the present disclosure provides a method, the method comprising: providing a filter medium comprising a functionalized nonwoven; passing a first fluid containing cells through the filter medium, wherein at least some of the cells are captured by the functionalized nonwoven; passing a second fluid, and optionally a third and/or fourth fluid through the filter medium, wherein the second and/or third and/or fourth fluid disrupts at least one of the captured cells; and recovering an intracellular biological product in the first and/or second and/or third and/or fourth fluid.

Various aspects and advantages of exemplary embodiments of the present disclosure have been summarized. The above Summary is not intended to describe each illustrated embodiment or every implementation of the present disclosure. Further features and advantages are disclosed in the embodiments that follow. The Detailed Description that follow more particularly exemplify certain embodiments using the principles disclosed herein.

### DETAILED DESCRIPTION

Before any embodiments of the present disclosure are explained in detail, it is understood that the invention is not limited in its application to the details of use, construction, and the arrangement of components set forth in the following description. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting and information that is relevant to a section heading may occur within or outside of that particular section.

The term “about” as used herein can allow for a degree of variability in a value or range. For example, within 10%, within 5%, or within 1% of a stated value or of a stated limit of a range and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less.

As used herein “layer” means a thickness of material the fluid to be processed passes through wherein the material in the layer is all formed from the same material. A layer can be a monolithic layer formed from a thickness of the same material. Or a layer can have one or more discrete plies of the same material stacked one on top of another within the layer to form its thickness. For example, a layer of common facial tissue is often a tissue paper material that is made from two individual plies of tissue paper placed in face to face contact and the two individual plies can be easily separated from each other as they are commonly held together by weak mechanical bonds in the form of crimp lines.

As used herein a “functionalized nonwoven” is a nonwoven that will attract target particles or molecules by attractive forces such as electrostatic forces due to the presence at the surfaces of that nonwoven of one or more of chemical moieties, ligands, or functional groups that are distinct from the materials forming the bulk of the nonwoven which are providing primarily its structural shape and integrity. The chemical moieties, ligands, or functional groups are specifically intended to attract the target particles or molecules to the surfaces of the functionalized nonwoven. Functionalized nonwovens may be created by coating or grafting a porous nonwoven with ligands, monomers, or polymers designed to molecularly attract the target particles or molecules. Alternatively, functionalized nonwovens may be created by the provision, in the formulation used to make such nonwovens, of surface modifying polymers or chemical moieties that become localized at the surfaces of the nonwoven during its formation, resulting in the presence on the surfaces of the nonwoven of chemical groups designed to attract the target particles or molecules. In some embodiments, the attractive force between the functional groups on the surfaces of the functionalized nonwoven are electrostatic forces, and the chemical moieties, ligands, or polymers present on the surfaces of the functionalized nonwoven are electrostatically charged. A functionalized nonwoven may have a positive charge and attract negatively charged particles, i.e. anion exchange chromatography, or the functionalized nonwoven may have a negative charge and attract positively charged particles, i.e. cation exchange chromatography. In other embodiments, the attractive forces may be Van der Waals forces, and the target particles or molecules are attracted to the functional groups on the functionalized nonwoven surfaces by mutual relative concentration or paucity of polarizable or hydrogen bonding moieties (i.e., hydrophobic interaction). Further, the attractive forces may include a combination of electrostatic and Van der Waals forces (i.e., mixed mode). Functionalized material suitable for the functionalized nonwovens in the charged depth filter device are made by Pall, Millipore, and Sartorious and sold under the following brands: Mustang® Q, NatriFlo® HD-Q and Sartobind® Q. Functionalized nonwovens suitable for use in the charged depth filter device can be nonwovens, membranes, or other suitable materials. A preferred functionalized nonwoven material is made by 3M Company and disclosed in U.S. Pat. No. 9,821,276 entitled “Nonwoven Article Grafted with Copolymer.” A preferred functionalized membrane is made 3M Company and disclosed in U.S. Pat. Nos. 9,650,470; and 10,017,461 entitled “Method of Making Ligand Functionalized Substrates.” All three mentioned patents are herein incorporated by reference in their entirety.

As used herein “osmotic potential” means the potential of water molecules to move from a hypotonic solution (more water, less solutes) to a hypertonic solution (less water, more solutes) across a semi permeable membrane. Osmotic potential can be calculated by using tils formula: Osmotic potential=−C×R×T, where C is the concentration of solute (i.e. sucrose, salt, etc.), R is universal gas constant (i.e. 8.314472 J K-1 mol-1), T is the absolute temperature.

### Method of Clarification

The present disclosure provides a method of harvesting biologics. The method includes providing a filter medium comprising a functionalized nonwoven. A first fluid containing cells can be passed through the filter medium. At least some of the cells can be captured and immobilized by the functionalized nonwoven. A second fluid, and optionally a third and/or fourth fluid can be passed through the filter medium after the first fluid containing cells is passed through the filter medium to disrupt the captured cells. After the captured cells are disrupted, the target product, for example, an intracellular biological product, can be recovered in the first and/or second and/or third and/or fourth fluid. Disruption of a cell involves a degradation of cell integrity so that the internal contents of the cell at least partially or completely issue from the cell into the fluid environment. This can involve a partial or a complete degradation of the cell membrane. This can involve an increase of the cell membrane's permeability. In one embodiment, cell disruption involves cell lysis.

The filter medium can include the filter disclosed in U.S. patent application Ser. No. 63/154,299 filed on Feb. 26, 2021 entitled Charged Depth Filter for Therapeutic Biotechnology Manufacturing Process and herein incorporated by reference in its entirety. In some embodiments, the filter medium can have at least two layers of a functionalized nonwoven, each layer having a same or different calculated pore size and a same or different dynamic charge capacity (MY DCC). In some embodiments, the filter medium can have multiple layers of functionalized nonwoven, each layer having a same or different calculated pore size and a same or different dynamic charge capacity. For example, the filter medium can have at least a first functionalized nonwoven layer having a first calculated pore size and a first dynamic charge capacity; and a second functionalized nonwoven layer having a second calculated pore size and a second dynamic charge capacity positioned after the first functionalized nonwoven layer in the direction of the biopharma feedstock flow; and wherein the first calculated pore size is greater than the second calculated pore size, and the first dynamic charge capacity is less than the second dynamic charge capacity.

In some embodiments, for the first functionalized nonwoven layer the first calculated pore size is from 40.8 μm to 65.0 μm and the first dynamic charge capacity is from 150 MY DCC mg/g to 300 MY DCC mg/g, and for the second functionalized nonwoven layer the second calculated pore size is from 5.0 μm to less than 40.8 μm and the second dynamic charge capacity is from greater than 300 MY DCC mg/g to 650 MY DCC mg/g.

In some embodiments, for the first functionalized nonwoven layer, the first calculated pore size is from 55.0 μm to 65.0 μm and the first dynamic charge capacity is from 150 MY DCC mg/g to 300 MY DCC mg/g, and for the second functionalized nonwoven layer the second calculated pore size is from 5.0 μm to less than 55.0 μm and the second dynamic charge capacity is from 300 MY DCC mg/g to 650 MY DCC mg/g.

In some embodiments, the filter medium can have a third functionalized nonwoven layer having a third calculated pore size and a second dynamic charge capacity positioned after the second functionalized nonwoven layer in the direction of the biopharma feedstock flow and the second calculated pore size is greater than the third calculated pore size, and the second dynamic charge capacity is less than the third dynamic charge capacity.

In some embodiments, for the first functionalized nonwoven layer, the first calculated pore size is from 40.8 μm to 65.0 μm and the first dynamic charge capacity is from 150 MY DCC mg/g to 300 MY DCC mg/g, and for the second functionalized nonwoven layer the second calculated pore size is from 20.6 μm to less than 40.8 μm and the second dynamic charge capacity is from greater than 300 MY DCC mg/g to 475 MY DCC mg/g, and for the third functionalized nonwoven layer the third calculated pore size is from 5.0 μm to less than 20.6 μm and the third dynamic charge capacity is from greater than 300 MY DCC mg/g to MY DCC 650 mg/g.

In some embodiments, for the first functionalized nonwoven layer the first calculated pore size is from 55.0 μm to 65.0 μm and the first dynamic charge capacity is from 150 MY DCC mg/g to 300 MY DCC mg/g, and for the second functionalized nonwoven layer the second calculated pore size is from 20.6 μm to less than 55.0 μm and the second dynamic charge capacity is from 200 MY DCC mg/g to 475 MY DCC mg/g, and for the third functionalized nonwoven layer the third calculated pore size is from 5.0 μm to less than 20.6 μm and the third dynamic charge capacity is from greater than 300 MY DCC mg/g to 650 MY DCC mg/g.

Identical layers may be repeated within the filter medium to increase capacity for specific debris sizes before the pore size and/or dynamic charge capacity is changed. The media stack of the charged depth filter can have 2, 3, 4, 5, 6, 7, 8, 9, or 10 or more layers depending on the construction, but will often have less than 25 layers.

In some embodiments, the first fluid can include cell culture feedstock, for example, mammalian cell cultures (e.g. Chinese hamster ovary (CHO) cells, Human embryonic kidney 293 (HEK-293) cells, baby hamster kidney (BHK21) cells, NSO murine myeloma cells, or PER. C6® human cells), insect and bacterial cell lines. In one embodiment, the first fluid can be a transfected cell culture. In one embodiment, the first fluid can be a HEK-293 cell culture that has been subjected to a triple plasmid transfection for the production of adeno associated virus.

In some embodiments, the second fluid can have an osmotic potential less than the osmotic potential of the first fluid, and the change in osmotic potential within the filter medium can disrupt captured cells. For example, the second fluid can have an osmotic potential of less than −500 J, less than −620 J, or less than −750 J, and the first fluid can have an osmotic potential of from −5 to −1 J. In some of these embodiments, the second fluid can have a conductivity less than a conductivity of the first fluid. For example, the second fluid can have a conductivity of less than 5 mS/cm and the first fluid can have a conductivity of 5 to 20 mS/cm. In these embodiments, the second fluid can be a sucrose solution. In some other embodiments, the second fluid can have a conductivity more than a conductivity of the first fluid. In these embodiments, the second fluid can be a salt solution, for example, NaCl solution, Phosphate Buffered Saline, Phosphate Buffer, Tris-HCl buffer, Tris-Acetate buffer, HEPES Buffer [4-(2-hydroxyethyl)-1-piperazine ethane sulfonic acid].

In some embodiments, the second fluid can have an osmotic potential more than the osmotic potential of the first fluid, and the change in osmotic potential within the filter medium can disrupt captured cells. For example, the second fluid can have an osmotic potential of from −5 J to 0 J and the first fluid can have an osmotic potential of from −5 J to −1 J. In some of these embodiments, the second fluid can have a conductivity less than a conductivity of the first fluid. In these embodiments, the second fluid can be water.

In some embodiments, the second fluid can be a surfactant or a chemical cell lysis agent. The surfactant or chemical cell lysis agent, can be any suitable surfactant, for example, non-ionic surfactants, cationic surfactants, a net charge of zero (zwitterionic detergents), and mixtures thereof. In some embodiments, the surfactant or chemical cell lysis agent can include Triton X1-100 or Tween 20, Tergitol NP9, and polysorbate.

In some embodiments, a conductivity of the third fluid is different than that of the first fluid and different than that of the second fluid. For example, the third fluid can have a conductivity ranging from 9 to 60 mS/cm, the second fluid can have a conductivity of less than 5 mS/cm and the first fluid can have a conductivity of 5 to 20 mS/cm. In some embodiments, the difference of conductivity between the first fluid and the third fluid is at least 2 mS/cm, 3 mS/cm, 5 mS/cm, 10 mS/cm, 20 mS/cm, 30 mS/cm, or 50 mS/cm.

In some embodiments, the third or fourth fluid can be a conductive salt solution with a conductivity ranging from 9 to 60 mS/cm. In some embodiments, the third or fourth fluid can be selected from NaCl solution, Phosphate Buffered Saline, Phosphate Buffer, Tris-HCl buffer, Tris-Acetate buffer, HEPES Buffer [4-(2-hydroxyethyl)-1-piperazine ethane sulfonic acid].

In some embodiments, the second fluid can have an osmotic potential less than the osmotic potential of the first fluid and have a conductivity less than a conductivity of the first fluid, the third fluid can have an osmotic potential more than the osmotic potential of the first fluid, and the fourth fluid can have an osmotic potential less than the osmotic potential of the first fluid and have a conductivity more than a conductivity of the first fluid. For example, the second fluid can be a sucrose solution, the third fluid can be water and the fourth fluid can be NaCl solution. In these embodiments, the first fluid, cell culture media as well as other soluble contaminants can flow through the filter medium comprising functionalized nonwoven resulting in concentration of the cells as they are captured. Application of a second fluid, hypertonic solution, such as 40 wt % sucrose or high salt solution, drives water out of the immobilized cells and serves to increase osmotic potential. Following an equilibration period in the hypertonic solution, the environment can be rapidly switched to hypotonic conditions without altering the cell concentration. The hypertonic solution, second fluid, can be drained from the functional nonwoven and a hypotonic medium, such as deionized water, can be applied to the cells captured on the functionalized nonwoven. The sudden change in environment can drive water into the cells, which increases their volume and leads to lysis. Following the lysis step, the fourth liquid can be applied to the functional nonwoven to recover residual product while retaining impurities (i.e. soluble and insoluble species) by the functionalized nonwoven. Capturing the cells/cell debris by charge can enable the fourth liquid to be applied while maintaining a low differential pressure across the nonwoven.

In some embodiments, the second fluid can have an osmotic potential more than the osmotic potential of the first fluid, and the third fluid can have an osmotic potential less than the osmotic potential of the first fluid and have a conductivity more than a conductivity of the first fluid. For example, the second fluid can be water and the third fluid can be NaCl solution. In these embodiments, there may be no need to pass the fourth fluid through the filter medium.

In some embodiments, the second fluid can be a surfactant, or a chemical cell lysis agent and the third fluid can have an osmotic potential less than the osmotic potential of the first fluid and have a conductivity more than a conductivity of the first fluid. For example, the second fluid can be Triton X1-100 or Tween 20 and the third fluid can be NaCl solution. In these embodiments, there may be no need to pass the fourth fluid through the filter medium.

In some embodiments, the second fluid can have an osmotic potential less than the osmotic potential of the first fluid and have a conductivity more than a conductivity of the first fluid. For example, the second fluid can be NaCl solution. In these embodiments, there may be no need to pass the third and/or fourth fluid through the filter medium.

The biological product clarified by the method of the present application can be an intracellular biological product, for example, adeno-associated virus (AAV) capsids, therapeutic/recombinant proteins, plasmids, DNA, RNA, viruses, virus like particles, exosomes, and mixtures thereof.

Current biological product harvest protocols, especially for intracellular biological products, often require the addition of a detergent followed by complex, multi-stage primary clarification strategies to process the generated lysate. The large and varied particle size distribution often presents a challenge to the conventional clarification approaches, leading to multistage filter configurations with large effective filter areas. Measures also need to be taken to remove the detergent in subsequent purification steps to ensure the final product is free of this contaminant.

The present method provides a differentiated approach: the lysis and clarification steps occur sequentially within a single device. The technology also makes available the use of osmotic pressure to lyse the cells, negating the need to add any additional reagents. For example, in some embodiments, the method involves establishing an osmotic pressure difference between the interior of the cell and the surrounding environment. Lysis can take place when the change in pressure occurs rapidly and a large pressure difference across the cell membrane is created. Process limitations, such as the time and challenges associated with buffer exchange (i.e. dilution volume and capital investment required for large-scale equipment such as a centrifuge) have limited osmotic lysis to small-scale applications. In addition, the current method can be specifically tailored to accommodate the demands posed by an in-situ cell lysis such as high cell loadings that generate large volumes of cell debris upon lysis. Rather than attempt to osmotically lyse cells in bulk solution, the cells can be chromatographically captured and concentrated through charge-based interactions on a functionalized nonwoven. The extracellular environment can then be easily manipulated to facilitate osmotic lysis. For example, the extracellular environment can be rapidly changed from hypertonic to hypotonic conditions to induce lysis of cells captured on the nonwoven. As lysis occurs, generated cell debris is simultaneously captured and retained by the functionalized nonwoven while the product of interest is collected in the flow through/filtrate. Application of functionalized nonwoven in this fashion makes osmotic lysis a viable candidate for harvesting intracellularly expressed biologics and mitigates challenges posed by other lytic strategies, such as the large dilutions associated with buffer exchange or the process complications. Additional clarification steps to remove debris generated during lysis can be eliminated using the method because the functionalized nonwoven retains debris generated during the cell disruption. In addition to retaining large scale, insoluble debris, the functionalized nonwoven can reduce the concentration of soluble impurities present in the lysate, such as DNA and host cell proteins. For example, the functionalized nonwoven can have the potential of reducing DNA concentrations in the first fluid below 10 ng/ml.

### Nonwoven

The nonwoven can be a nonwoven web which may include nonwoven webs manufactured by any of the commonly known processes for producing nonwoven webs. As used herein, the term “nonwoven web” refers to a fabric that has a structure of individual fibers or filaments which are randomly and/or unidirectionally interlaid in a mat-like fashion. For example, the fibrous nonwoven web can be made by carded, air laid, wet laid, spunlaced, spunbonding, electrospinning or melt-blowing techniques, such as melt-spun or melt-blown, or combinations thereof. Spunbonded fibers are typically small diameter fibers that are formed by extruding molten thermoplastic polymer as filaments from a plurality of fine, usually circular capillaries of a spinneret with the diameter of the extruded fibers being rapidly reduced. Melt-blown fibers are typically formed by extruding the molten thermoplastic material through a plurality of fine, usually circular, die capillaries as molten threads or filaments into a high velocity, usually heated gas (e.q. air) stream which attenuates the filaments of molten thermoplastic material to reduce their diameter. Thereafter, the meltblown fibers are carried by the high velocity gas stream and are deposited on a collecting surface to form a web of randomly distributed meltblown fibers. Any of the non-woven webs may be made from a single type of fiber or two or more fibers that differ in the type of thermoplastic polymer and/or thickness.

Suitable polyolefins for making the nonwoven web include, but are not limited to, polyethylene, polypropylene, poly(1-butene), copolymers of ethylene and propylene, alpha olefin copolymers (such as copolymers of ethylene or propylene with 1-butene, 1-hexene, 1-octene, and 1-decene), poly(ethylene-co-1-butene), poly(1-methylpentene) and poly(ethylene-co-1-butene-co-1-hexene). Preferably the nonwoven substrate is a polypropylene.

Further details on the manufacturing method of nonwoven webs of this invention may be found in Wente, Superfine Thermoplastic Fibers, 48 INDUS. ENG. CHEM. 1342(1956), or in Wente et al. Manufacture of Superfine Organic Fibers, (Naval Research Laboratories Report No. 4364, 1954). Useful methods of preparing the nonwoven substrates are described in U.S. RE39,399 (Allen), U.S. Pat. No. 3,849,241 (Butin et al.), U.S. Pat. No. 7,374,416 (Cook et al.), U.S. Pat. No. 4,936,934 (Buehning), and U.S. Pat. No. 6,230,776 (Choi).

### Functionalized Nonwoven

The functionalized nonwoven can include the nonwoven substrate discussed above and a grafted copolymer comprising interpolymerized monomer units, at least one of which is cationic or can be made cationic in solution of appropriate pH (“cationically ionizable”). A suitable functionalized nonwoven is disclosed in U.S. Pat. No. 9,821,276 entitled “Nonwoven Article Grafted With Copolymer” issued on Nov. 21, 2017 and herein incorporated by reference.

The cationic or cationically ionizable monomer can include quaternary ammonium-containing monomers and tertiary amine-containing monomers. One or more than one cationic or cationically ionizable monomer may be used. Monomers typically contain polymerizable functionalities as well as cationic or cationically ionizable groups. In certain monomers, the polymerizable group and the cationic group may be the same group. Polymerizable groups include vinyl, vinyl ether, (meth)acryloyl, (meth)acrylamido, allyl, cyclic unsaturated monomers, multifunctional monomers, vinyl esters, and other readily polymerizable functional groups.

Useful (meth)acrylates include, for example, trimethylaminoethylmethacrylate, trimethylaminoethylacrylate, triethylaminoethylmethacylate, triethylaminoethylacrylate, trimethylaminopropylmethacrylate, trimethylaminopropylacrylate, dimethylbutylaminopropylmethacrylate, diethylbutylaminopropylacrylate, 2-(dimethylamino)ethyl methacrylate, 2-(diethylamino)ethyl methacrylate, 2-(dimethylamino)ethyl acrylate, 2-(diethylamino)ethyl acrylate, and 3-(dimethylamino)propyl acrylate.

Exemplary (meth)acrylamides include, for example, 3-(trimethylamino) propylmethacrylamide, 3-(triethylamino)propylmethacrylamide, 3-(ethyldimethylamino)propylmethacrylamide, and n-[3-(dimethylamino)propyl]methacrylamide. Preferred quaternary salts of these (meth)acryloyl monomers include, but are not limited to, (meth)acrylamidoalkyltrimethylammonium salts e.g., 3-methacrylamidopropyltrimethylammonium chloride and 3-acrylamidopropyltrimethylammonium chloride) and (meth)acryloxyalkyltrimethylammonium salts (e.g., 2-acryloxyethyltrimethylammonium chloride, 2-methacryloxyethyltrimethylammonium chloride, 3-methacryloxy-2-hydroxypropyltrimethylammonium chloride, 3-acryloxy-2-hydroxypropyltrimethylammonium chloride, and 2-acryloxyethyltrimethylammonium methyl sulfate).

The grafted copolymer further comprises optional monomer units which can be co-polymerized with the cationic or cationically ionizable monomer(s). While it may be possible to ionize these monomers under certain conditions, they are typically not charged; they are neutral (“neutral monomer”). These neutral monomers have a polymerizable group for use during the graft polymerization. This polymerizable group may be the same as, or different from, the polymerizable group(s) on the cationic or cationically ionizable monomer(s). There may be one or more than one neutral monomer.

Neutral monomers may have a functional group or more than one functional group in addition to the polymerizable group. In the case of neutral monomers with more than one functional group, those functional groups may be the same or different. Some functional groups may enable the neutral monomer to dissolve or disperse in water. Some functional groups may be hydrophilic after polymerizing. Useful functional groups include hydroxyl, alkyl, aryl, ether, ester, epoxy, amide, isocyanate, or, cyclic functional groups. Neutral monomers may contain spacer groups between the polymerizing group and the functional group. Neutral monomers may contain oligomeric or polymeric functional group(s). In some embodiments, the polymerizing group and the functional group may be the same group.

Examples of epoxy containing neutral monomers include glycidyl(meth)acrylate, thioglycidyl(meth)acrylate, 3-(2,3-epoxypropoxy)phenyl(meth)acrylate, 2-[4-(2,3-epoxypropoxyl)phenyl]-2-(4-(meth)acryloyloxy-phenyl)propane, 4-(2,3-epoxypropoxyl)cyclohexyl(meth)acrylate, 2,3-epoxycyclohexyl(meth)acrylate, and 3,4-epoxycyclohexyl(meth)acrylate, and combinations thereof. Examples of hydroxyl containing monomers include N-hydroxyethyl(meth)acrylate, poly(ethylene glycol)(meth)acrylates, poly(propylene glycol)(meth)acrylates, N-hydroxyethyl(meth)acrylamide, 2-hydroxypropyl(meth)acrylamide, N-hydroxypropyl(meth)acrylate, 2-hydroxy-3-phenoxypropyl(meth)acrylate, and combinations thereof. Examples of suitable amide monomers include N-vinyl caprolactam, N-vinyl acetamide, N-vinyl pyrrolidone, (meth)acrylamide, mono- or di-N-alkyl substituted acrylamide, and combinations thereof. Examples of suitable ether monomers include poly(ethylene glycol)(meth)acrylates, poly(propylene glycol)(meth)acrylates, 2-ethoxyethyl (meth)acrylate, ethylene glycol methyl ether (meth)acrylate, N-3-methoxypropyl(meth)acrylamide, di(ethylene glycol)methyl ether(meth)acrylate, poly(ethylene glycol)phenyl ether (meth)acrylate, 2-pheoxyethyl(meth)acrylate, other alkyl ether (meth)acrylates and alkyl ether (meth)acrylamides, tetrahydrofurfuryl(meth)acrylate, and combinations thereof.

The process of preparing the functionalized nonwoven includes the steps of providing a nonwoven substrate, exposing the nonwoven substrate to ionizing radiation in an inert atmosphere, and subsequently contacting the exposed substrate with a solution or suspension comprising the grafting monomers to graft polymerize said monomers to the nonwoven substrate.

In the first step the nonwoven substrate is exposed to ionizing radiation in an inert atmosphere. Exemplary forms of ionizing radiation include electron beam (e-beam), gamma, x-ray, and other forms of electromagnetic radiation. The inert atmosphere is generally an inert gas such as nitrogen, carbon dioxide, helium, argon, etc. with a minimal amount of oxygen. Doses delivered by the ionizing radiation source may happen in a single dose or may be in multiple doses which accumulate to the desired level. One or more layers of nonwoven substrates may be subjected to the ionizing radiation

After the irradiation step, the irradiated nonwoven substrate is contacted with the aqueous monomer solution or suspension. “Contacted” means bringing the irradiated nonwoven substrate into contact with the monomer solution or suspension. It can also be described as the irradiated nonwoven substrate being saturated, imbibed, or coated with monomer solution. The monomer solution may only partially fill the void volume of the nonwoven substrate, or much more solution can be contacted to the nonwoven substrate than is necessary to fully fill the void volume. The monomer contact step also occurs in an inert atmosphere. This atmosphere may be the same as, or different from, the atmosphere in the chamber where the substrate is irradiated. The chamber may be the same as, or different from, the chamber where the substrate is irradiated. The monomer solution remains in contact with the nonwoven substrate for a time sufficient for the graft polymerization with some, most, or substantially all of the monomers in the monomer solution. Once the nonwoven substrate has been contacted for a desired period of time, the nonwoven substrate bearing grafted polymer may be removed from the inert atmosphere.

### Nonfunctionalized and Functionalized Nonwoven Parameters

Properties of interest for nonfunctionalized nonwovens and functionalized nonwovens (e.g. copolymer grafted nonwovens) include basis weight, effective fiber diameter (EFD), solidity, and pore size. They can be determined for the nonwoven prior to functionalization or after being functionalized.

The fibers of the nonfunctionalized nonwoven substrate typically have an effective fiber diameter of about 3 to 20 micrometers. The nonfunctionalized substrate preferably has basis weight in the range of about 10 to 400 g/m², more preferably about 80 to 250 g/m². The average thickness of the nonfunctionalized substrate is preferably about 0.1 to 10 mm, and more preferably about 0.25 to 5 mm.

The functionalized or nonfunctionalized nonwoven's loft is measured by solidity, a parameter that defines the solids fraction in a volume of web. Lower solidity values are indicative of greater web loft.

Solidity is a unitless fraction typically represented by a:

α=m_{f}÷(ρ_{f}×L_{nonwoven})

Basis weight, m_{f}, is the mass (functionalized or nonfunctionalized) per surface area and ρ_{f} is the fiber density (functionalized or nonfunctionalized). L_{nonwoven} is the nonwoven thickness (functionalized or nonfunctionalized). Solidity can be determined for the nonwoven prior to or after functionalization.

Fiber density (ρ_{f}) of copolymer grafted fibers after functionalization is determined by Method A in the Examples described below. Fiber density of copolymer grafted fibers after functionalization can also be determined by a modified version of Method A in which the substrate and copolymer component molar ratios are all obtained from solid state Carbon-13 NMR measurements and the molar ratios are converted to weight ratios. When a nonwoven substrate contains mixtures of two or more kinds of fibers, the individual solidities are determined for each kind of fiber using the same L_{nonwoven} and these individual solidities are added together to obtain the web's solidity, a.

Effective fiber diameter (EFD) is the apparent diameter of the fibers in a nonwoven fibrous web determined by an air permeation test in which air at 1 atmosphere and room temperature is passed at a face velocity of 5.3 cm/sec through a web sample of known thickness, and the corresponding pressure drop is measured. Based on the measured pressure drop, the effective fiber diameter is calculated set forth in Davies, C. N., “The Separation of Airborne Dust and Particles”, Institution of Mechanical Engineers, London, Proceedings 1B, 1952. EFD can be determined for the nonwoven prior to or after functionalization.

The calculated pore size is related to the arithmetic median fiber diameter and web solidity and is determined by the following formula: where D is the calculated pore size, d_{f} is arithmetic median fiber diameter, and a is the web solidity.

$$D = {d}_{f} \{ {( \frac{2 α}{π} )}^{- 1 / 2} - 1 \}$$

Calculated pore size can be determined for the nonwoven prior to or after functionalization. The nonwoven substrate, prior to functionalization, preferably has a calculated pore size of 1-50 micrometers.

The Dynamic Charge Capacity (DCC) of the functionalized nonwoven substrate is determined using a metanil yellow challenge solution using Method B in the Examples and is reported as the MY DCC (Metanil Yellow Dynamic Charge Capacity).

The following working examples are intended to be illustrative of the present disclosure and not limiting.

### Examples

Objects and advantages of this invention are further illustrated by the following examples, but the particular materials and amounts thereof recited in these examples, as well as other conditions and details, should not be construed to unduly limit this invention.

Conductivity measurements of cell culture and eluant fluids were conducted using an Accumet Excel XL50 conductivity meter (Fisher Scientific, Hampton, NH) at ambient temperature (about 20° C.).

### Grafting Solution

TABLE 1
Materials

| Description (abbreviation) | Source |
|---|---|
| 3-methacrylamidopropyltrimethylammonium | Evonik Corporation, |
| chloride (MAPTAC) (50 weight % in water) | Parsippany, NJ |
| [CAS No. 51410-72-1] | |
| N-vinyl-2-pyrrolidone (NVP) | Ashland Chemicals, |
| | Covington, KY |
| Glycidyl methacrylate (GMA) | Dow Chemical, |
| | Midland, MI |

The Grafting Solution was prepared as a monomer solution containing 12.2% NVP, 4.4% GMA, and 9.7% MAPTAC all by weight in deionized water.

### Method A. Determination of Basis Weight, Effective Fiber Diameter (EFD), Solidity, and Pore Size of Functionalized Nonwovens

Basis weight, EFD, solidity, and pore size measurements of the functionalized nonwovens were determined according to the following procedure. Sample discs (13.33 cm diameter) were punched from functionalized nonwoven sheets and then individually rinsed by submerging each disc in a 2 L bath of deionized water for 15 minutes. The rinse procedure was repeated three additional times using fresh deionized water for each rinse step. Each rinsed disc was dried in an oven at 70° C. for at least 4 hours. During the drying step, a weight (about 100 g) was placed on top of each disc to prevent edge curling. The resulting dried functionalized nonwoven samples were characterized (basis weight, EFD, solidity, pore size) according to the methods and equations described above. For each measurement or calculation, the results were reported as the mean value of three independent trials (n=3) with the calculated standard deviation (SD).

For the solidity (a) equation, the fiber density (ρ_{f}) measurement was determined as the sum of the density of the polypropylene substrate (0.91 g/cm³) and the density of the grafted copolymer (1.07 g/cm³) adjusted by the weight ratio of the polypropylene substrate and grafted copolymer of the test sample (Equation 1). The polypropylene substrate and copolymer weight ratios were determined by comparing the basis weight of the nonwoven before the grafting step to the basis weight of the corresponding dried functionalized nonwoven.

The density of the grafted copolymer (D_{GCP}) was determined by first using solid state ¹³C NMR (ssNMR) to measure the mol % of monomer components (NVP, MAPTAC, GMA) of the grafted copolymer; converting the mol % values to weight % (wt. %) values. The density value of each monomer component (monomer densities: D_{NVP}=1.04 g/cm³, D_{MAPTAC}=1.067 g/cm³, D_{GMA}=1.07 g/cm³) was adjusted (multiplied) by the corresponding component wt. % value, and the resulting three adjusted density values were summed (Equation 2).

Fiber density (ρ_{f})=(0.91×wt. % polypropylene)+(1.05×wt. % grafted copolymer) Equation 1:

D_{GCP}=(D_{NVP}×wt. %_{NVP})+(D_{MAPTAC}×wt. %_{MAPTAC})+(D_{GMA}×wt. %_{GMA}) Equation 2:

### Method B. Determination of the Metanil Yellow Dynamic Charge Capacity (MY DCC) of Functionalized Nonwovens

Functionalized nonwoven discs were prepared for testing according to Method A. The dynamic charge capacity of a disc was determined using the charged organic dye metanil yellow as the target molecule in the challenge solution. The challenge solution used had a metanil yellow concentration of 160 mg/L (160 ppm). The challenge solution was prepared by dissolving 3.2 g of metanil yellow, 93.98 g of sodium phosphate dibasic anhydrous, 46.64 g of sodium phosphate monobasic monohydrate and 163.63 g of NaCl in 20 L of deionized water. The challenge solution was used within 2 days of preparation. If needed, the amount of metanil yellow reagent used to prepare the challenge solution was adjusted based on the purity of the regent so that the challenge solution contained 160 ppm of metanil yellow. An analytical standard grade metanil yellow (>98.0%, product #44426 from the Sigma-Aldrich Company, St. Louis, MO) was used to calibrate reagent purity. A buffer solution for pre-conditioning the test assembly was also prepared having the same formulation as the challenge solution except that metanil yellow was not included.

The filtration test assembly contained a clear, polycarbonate body section (47 mm inner diameter) with a screw-on cap attached to the top of the body section. The cap contained an inlet port and a vent port. The bottom of the body section contained an outlet port with a stopcock. A pressure sensor was placed upstream of the inlet port. A polyamide membrane (0.2 micrometer grade) was placed at the bottom of the body section. A stack containing two functionalized nonwoven discs (each 47 mm diameter and punched from a disc prepared according to Method A) was placed in the assembly on top of the membrane. In the assembly, the nonwoven discs were sandwiched between two PTFE seal rings each containing a knife edge on the inner diameter to bite into the nonwoven. The resulting sub-assembly was secured in place using an O-ring. The frontal surface area of the disc stack was 0.00097 m². The cap was attached to the body section and a PendoTech normal flow filtration system (PendoTech Company, Princeton, NJ) was connected to the inlet port. A Hach Model 2100AN turbidimeter (Hach Company, Loveland, CO) with a 455 nm light filter, and a flow-through cell was connected to the output port and used to measure the metanil yellow concentration in the filtrate. Metanil yellow solutions with concentration of 0.8 ppm, 4 ppm, and 8 ppm were prepared as test standards. The end point of the charge capacity measurement was set at the 5% breakthrough (8 ppm) of metanil yellow solution. The fluid flow rate was 15 mL/minute. Prior to pumping the challenge solution, the preconditioning buffer was flushed through the assembly for about 5 minutes.

The volume of challenge solution that passed through the test assembly up to the end point (i.e. the breakthrough volume) was measured and the dynamic charge capacity (mg/g) of the functionalized nonwoven sample was calculated according to Equation 3. For each functionalized nonwoven, MY DCC was reported as the mean value from three independent trials (n=3) with the calculated standard deviation (SD).

$$MY DCC = \frac{Breakthrough volume ( mL ) \times Mentanil Yellow concentration ( mg / mL )}{Total Basis weight of FNW ( g / m 2 ) \times FNW disc frontal surface area ( m 2 )} \qquad Equation 3$$

### Method C. Preparation of AAV2 Transfected Cell Culture Fluid

HEK293-F cells suspended in Gibco LV-MAX Production Medium (Thermo Fisher Scientific, Waltham, MA) were grown in an incubator using 2.8 L shaker flasks with shaking at a constant rate of 90 rpm (revolutions per minute). The incubator was maintained at 37° C. with 8% CO₂. When the cell density reached approximately 2×10⁶ cells/mL, a transfection cocktail was prepared and administered to the shaker flask.

The transfection cocktail consisted of the three plasmids pAAV2-RC2 Vector (Part No. VPK-422), pHelper Vector (Part No. 340202), and pAAV2-GFP Control Vector (Part No. AAV2-400) (all plasmids obtained from Cell Biolabs, San Diego, CA), and FECTOVIR-AAV2 transfection reagent (Polyplus Transfection, New York, NY). The transfection cocktail was prepared by first adding equimolar amounts of all three plasmids and the total plasmid amount was adjusted to be one microgram of plasmid mixture per million HEK cells used for transfection. Next, DMEM (Dulbecco's Modified Eagle Medium, obtained from Thermo Fisher Scientific) was added to the cocktail so that a final concentration of 5% DMEM (volume/volume) was achieved after adding the cocktail to the cell culture flask (i.e. volume/volume calculations for DMEM were adjusted based on the total cell culture volume). After the addition of DMEM, the cocktail was mixed and then one microliter of FectoVIR-AAV2 transfection reagent was added for every microgram of the plasmid mixture in the cocktail. The cocktail was gently mixed followed by incubation at room temperature for 45 minutes. Following the incubation step, the completed transfection cocktail was gently mixed and then added dropwise to the flask containing the cell culture. After addition of the transfection cocktail, the cells were grown in the incubator (37° C. with 8% CO₂) for 72-96 hours to induce the production of AAV2.

Cell viability was measured using a hemocytometer. The harvested cell culture fluid was mixed with 10% (volume/volume) trypan blue solution and then loaded to a disposable hemocytometer. Viable and dead cells were counted under microscopy.

The turbidity measurements of the transfected cell cultures and the filtrates (after filtration of a cell culture fluid through a filtration capsule) were determined in nephelometric turbidity units (NTU) using an ORION AQ4500 turbidity meter (Thermo Fisher Scientific).

AAV2 transfected cell cultures used in the examples had conductivity values of about 9 to 10 mS/cm, cell density values of about 3×10⁶ to 7×10⁶ cells/mL, cell viability values of about 75% to 90% at the time of harvest, and turbidity values of about 270 to 540 NTU.

### Preparation of Functionalized Nonwoven A (FNW-A)

A nonfunctionalized melt-blown polypropylene microfiber nonwoven web (having an effective fiber diameter (EFD) of 16 micrometers, basis weight of 200 grams per square meter (gsm), solidity of 10%, and calculated average pore size of 47.4 micrometers) was grafted with nitrogen purged Grafting Solution. The nonwoven substrate was unwound and passed through an electron beam (Electrocure, from Energy Science, Inc, Wilmington, MA) set to a potential of 300 kV and to deliver a total dose of 7 Mrad. The environment in the electron beam chamber was purged with nitrogen. The web was then conveyed directly into a nitrogen-purged saturation step with the monomer solution. The web was then wound up within the purged atmosphere. The web was left in the purged atmosphere for a minimum of 60 minutes, after which it was exposed to air. The web was then unwound and conveyed into a tank of deionized water for about 8 minutes at a speed of 10 feet per minute. After exiting the tank, the web was flushed multiple times by passing an aqueous salt solution (NaCl) through the web using a vacuum belt. A small amount of glycerin was added to the aqueous salt solution in the final flushing step. The unwound web was dried until the moisture content of the web was less than 14% by mass. The web was then wound up on a spindle. The grafted article was labeled as Functionalized Nonwoven A (FNW-A). The properties of FNW-A are reported in Table 2. Discs of FNW-A (2.54 cm diameter) were punched from the web.

### Preparation of Functionalized Nonwoven B (FNW-B)

A nonfunctionalized melt-blown polypropylene microfiber nonwoven web (having an effective fiber diameter of 14 micrometers, basis weight of 200 gsm, solidity of 10%, calculated average pore size of 41.5 micrometers) was grafted using same procedure described for FNW-A. The grafted article was labeled as Functionalized Nonwoven B (FNW-B). The properties of FNW-B are reported in Table 2. Discs of FNW-B (2.54 cm diameter) were punched from the web.

### Preparation of Functionalized Nonwoven C (FNW-C)

A nonfunctionalized melt-blown polypropylene microfiber nonwoven web (having an effective fiber diameter of 12 micrometers, basis weight of 200 gsm, solidity of 10%, calculated average pore size of 35.6 micrometers) was grafted using same procedure described for FNW-A. The grafted article was labeled as Functionalized Nonwoven C (FNW-C). The properties of FNW-C are reported in Table 2. Discs of FNW-C (2.54 cm diameter) were punched from the web.

### Preparation of Functionalized Nonwoven D (FNW-D)

A nonfunctionalized melt-blown polypropylene microfiber nonwoven web (having an effective fiber diameter of 10 micrometers, basis weight of 200 gsm, solidity of 10%, calculated average pore size of 29.6 micrometers) was grafted using same procedure described for FNW-A. The grafted article was labeled as Functionalized Nonwoven D (FNW-D). The properties of FNW-D are reported in Table 2. Discs of FNW-D (2.54 cm diameter) were punched from the web.

### Preparation of Functionalized Nonwoven E (FNW-E)

A nonfunctionalized melt-blown polypropylene microfiber nonwoven web (having an effective fiber diameter of 8 micrometers, basis weight of 200 gsm, solidity of 10%, calculated average pore size of 23.7 micrometers) was grafted using same procedure described for FNW-A. The grafted article was labeled as Functionalized Nonwoven E (FNW-E). The properties of FNW-E are reported in Table 2. Discs of FNW-E (2.54 cm diameter) were punched from the web.

### Preparation of Functionalized Nonwoven F (FNW-F)

A nonfunctionalized melt-blown polypropylene microfiber nonwoven web (having an effective fiber diameter of 6 micrometers, basis weight of 200 gsm, solidity of 10%, calculated average pore size of 17.8 micrometers) was grafted using same procedure described for FNW-A. The grafted article was labeled as Functionalized Nonwoven F (FNW-F). The properties of FNW-F are reported in Table 2. Discs of FNW-F (2.54 cm diameter) were punched from the web.

### Preparation of Functionalized Nonwoven G (FNW-G)

A nonfunctionalized melt-blown polypropylene microfiber nonwoven web (having an effective fiber diameter of 4.2 micrometers, basis weight of 100 gsm, solidity of 8.2%, calculated average pore size of 14.2 micrometers) was grafted using same procedure described for FNW-A. The grafted article was labeled as Functionalized Nonwoven G (FNW-G). The properties of FNW-G are reported in Table 2. MY DCC was determined by Method B using 4 discs of functionalized nonwoven instead of 2 discs. Discs of Functionalized Nonwoven G (2.54 cm diameter) were punched from the web.

TABLE 2
Properties of Functionalized Nonwovens A-G

| | | | | | Water | |
|---|---|---|---|---|---|---|
| | EFD | Basis Wt. | Solidity | Pore Size | Permeable | MY DCC |
| FNW | (micrometers) | (gsm) | (%) | (mm) | FNW | (mg/g) |
| A | 23.5 ± 0.2 | 267.1 ± 6.5 | 13.2 ± 0.7 | 57.7 ± 1.5 | Yes | 169.4 ± 25.8 |
| B | 21.6 ± 0.3 | 302.0 ± 5.1 | 14.2 ± 0.3 | 50.5 ± 0.6 | Yes | 165.0 ± 2.6 |
| C | 18.9 ± 1.2 | 272.7 ± 5.3 | 13.5 ± 0.6 | 45.6 ± 3.0 | Yes | 291.7 ± 20.4 |
| D | 14.6 ± 0.1 | 304.2 ± 3.3 | 14.4 ± 0.3 | 33.7 ± 0.4 | Yes | 316.0 ± 12.8 |
| E | 12.1 ± 0.3 | 356.6 ± 3.4 | 16.3 ± 0.7 | 25.5 ± 0.3 | Yes | 365.3 ± 10.5 |
| F | 9.1 ± 0.3 | 355.8 ± 11.4 | 17.8 ± 0.6 | 17.9 ± 0.2 | Yes | 407.4 ± 26.6 |
| G | 8.1 ± 0.2 | 318.5 ± 6.3 | 24.6 ± 0.1 | 12.4 ± 0.3 | No | 443.2 ± 28.1 |

### Example 1 (Ex. 1)

A plastic filtration capsule was used. The capsule consisted of a sealed, circular housing. The capsule housing was prepared from two halves (upper and lower halves) which were mated and sealed together at the perimeter after the filtration elements were inserted in the internal cavity of the lower housing. Fluid inlet and vent ports were located on the upper portion of the housing and a fluid outlet port was located on the lower portion of the housing. The outlet port was centered in the middle of the lower housing surface.

Two discs (27 mm diameter) of TYPAR 3161L polypropylene spunbond nonwoven (10 mil thick, obtained from Fiberweb, Inc., Old Hickory, TN) were placed in the bottom of the lower housing. A single disc (27 mm diameter) of a MICRO-PES Flat Type 2F polyethersulfone membrane with a 0.2 micrometer nominal pore size (obtained from the 3M Company) was placed on top of the nonwoven layer. The nonwoven and membrane layers were ultrasonically welded at the margins to the bottom inner surface of the lower housing. A stack of four functionalized nonwoven layers (27 mm diameter) was then placed on top of the membrane. The stack included two discs of Functionalized Nonwoven C, one disc of functionalized nonwoven E, and one disc of Functionalized Nonwoven F. The orientation of discs from capsule inlet to outlet was FNW-C/FNW-C/FNW-E/FNW-F. A polypropylene spacer ring (25.4 mm OD, 21.84 mm ID, 50 mil thick) was inserted between the second and third nonwoven layers. The upper and lower housings were mated together and ultrasonically welded using a Branson 20 kHz Ultrasonic welder (Model 2000xdt, Emerson Electric Company, St. Louis, MO) to form a finished filter capsule.

The overall outer diameter of the finished capsule was about 4.3 cm and the overall height including inlet, outlet, and vent ports was about 5.9 cm. The effective filtration area of the capsule was 3.2 cm² and the bed volume of the nonwoven media was 2.1 mL.

### Example 2 (Ex. 2)

The same procedure as described in Example 1 for preparing a filtration capsule was followed with the exception that a different stack of nonwoven layers was used. The stack included one disc of Functionalized Nonwoven C, one disc of Functionalized Nonwoven D, one disc of functionalized nonwoven E, and one disc of Functionalized Nonwoven F. The orientation of discs from capsule inlet to outlet was FNW-C/FNW-D/FNW-E/FNW-F. A polypropylene spacer ring (25.4 mm OD, 21.84 mm ID, 50 mil thick) was inserted between the second and third nonwoven layers.

### Example 3 (Ex. 3)

The same procedure as described in Example 1 for preparing a filtration capsule was followed with the exception that a different stack of nonwoven layers was used. The stack included two discs of Functionalized Nonwoven A, one disc of functionalized nonwoven E, and one disc of Functionalized Nonwoven F. The orientation of discs from capsule inlet to outlet was FNW-A/FNW-A/FNW-E/FNW-F. A polypropylene spacer ring (25.4 mm OD, 21.84 mm ID, 50 mil thick) was inserted between the second and third nonwoven layers.

### Example 4 (Ex. 4)

The same procedure as described in Example 1 for preparing a filtration capsule was followed with the exception that a different stack of nonwoven layers was used. The stack included three discs of Functionalized Nonwoven C, one disc of functionalized nonwoven E, and one disc of Functionalized Nonwoven F. The orientation of discs from capsule inlet to outlet was FNW-C/FNW-C/FNW-C/FNW-E/FNW-F. A polypropylene spacer ring (25.4 mm OD, 21.84 mm ID, 50 mil thick) was inserted between the third and fourth nonwoven layers.

### Example 5 (Ex. 5)

The same procedure as described in Example 1 for preparing a filtration capsule was followed with the exception that a different stack of nonwoven layers was used. The stack included one disc of Functionalized Nonwoven A, three discs of functionalized nonwoven C, and one disc of Functionalized Nonwoven F. The orientation of discs from capsule inlet to outlet was FNW-A/FNW-C/FNW-C/FNW-C/FNW-F. A polypropylene spacer ring (25.4 mm OD, 21.84 mm ID, 50 mil thick) was inserted between the third and fourth nonwoven layers.

### Example 6 (Ex. 6). Osmotic Lysis

A reservoir containing AAV2 transfected cell culture was connected to the inlet port of a capsule prepared according to Ex. 1 using flexible tubing. A Masterflex L/S peristaltic pump (Masterflex, Vernon Hills, IL) was used to pump the cell culture fluid through the capsule at a constant flux of 200 LMH and throughput of 100 L/m². The cell culture in the reservoir was stirred throughout the procedure. The resulting filtrate was collected and analyzed for AAV2 content (number of AAV2 capsids in the filtrate) using a ProGen AAV2 Xpress ELISA kit (obtained from American Research Products, Inc., Waltham, MA) according to the manufacturer's instructions.

After loading the capsule with the cell culture, three separate fluids were sequentially pumped through the capsule via the inlet port. The first fluid was an aqueous hypertonic sucrose solution (40 wt. %). Approximately 54 L/m² of the sucrose solution was pumped through the capsule at a constant flux of 200 LMH. The pump was then turned off to allow the functionalized filtration discs containing bound HEK293 cells to equilibrate with the sucrose solution remaining in the capsule for 15 to 30 minutes. Following the equilibration period, deionized water (62.5 L/m²) was pumped through the capsule at a constant flux of 200 LMH. The pump was again turned off to allow the functionalized filtration discs containing bound HEK293 cells to equilibrate with the deionized water remaining in the capsule for 15 to 30 minutes. Following the equilibration period, a hypertonic aqueous NaCl solution (400 mM, 40 mS/cm) was pumped through the capsule at a constant flux of 200 LMH and throughput of 62.5 L/m² for a final elution of AAV2 capsids from the functionalized nonwoven discs.

After administration of each of the three fluids, the collected filtrate was removed and analyzed for AAV2 capsid content (number of AAV2 capsids in each filtrate). During the procedure, a new collection vessel was used to collect each filtrate sample. The AAV2 content of each filtrate was determined using a ProGen AAV2 Xpress ELISA kit. The results are presented in Table 3.

TABLE 3
AAV2 Content of Recovered Filtrates
for the Procedure of Example 6

| | AAV2 Capsids |
|---|---|
| Filtrate Sample | in Filtrate |
| Cell Culture Flow Through | 5.58 × 10¹⁰ |
| Fluid 1 (sucrose | 5.81 × 10¹⁰ |
| solution, 40 wt. %) | |
| Fluid 2 (deionized water) | 5.84 × 10¹⁰ |
| Fluid 3 (aqueous | 1.01 × 10¹³ |
| NaCl solution, 400 mM) | |

### Example 7 (Ex. 7)

The same procedure as reported in Example 6 was followed with the exception that the cell culture was loaded at a constant flux of 200 LMH and throughput of 300 L/m². The results are presented in Table 4.

TABLE 4
AAV2 Content of Recovered Filtrates
for the Procedure of Example 7

| | AAV2 Capsids |
|---|---|
| Filtrate Sample | in Filtrate |
| Cell Culture/Flow Through | 1.07 × 10¹¹ |
| Fluid 1 (sucrose | 3.25 × 10¹⁰ |
| solution, 40 wt. %) | |
| Fluid 2 (deionized water) | 3.25 × 10¹⁰ |
| Fluid 3 (aqueous | 1.21 × 10¹³ |
| NaCl solution, 400 mM) | |

### Comparative Example A (CEx. A). Alternative Detergent Lysis Procedure

A 2.8 L shaker flask from the set used in the preparation of AAV2 transfected cell culture (Method C procedure) was charged with TRITON X-100 detergent (obtained from the Promega Corporation, Madison, WI) to achieve a final detergent concentration of 0.1 wt. %. To lyse the cells, the flask was placed on a shaker table in an incubator (set at 37° C., 8% CO₂) and shaken at 90 rpm for 2 hours. Next, the lysed cell culture fluid was clarified using the following procedure.

A reservoir containing the lysed cell culture fluid was connected to the inlet port of a capsule prepared according to Ex. 1 using flexible tubing. A Masterflex L/S peristaltic pump was used to pump the fluid through the capsule at a constant flux of 200 LMH and throughput of 100 L/m². The lysed cell culture fluid in the reservoir was stirred throughout the procedure. The resulting filtrate was collected and analyzed for AAV2 content using a ProGen AAV2 Xpress ELISA kit. A total of 4.45×10¹⁰ AAV2 capsids were recovered in the filtrate. The number of capsids recovered by this procedure was about 200-fold less than recovered using the procedure of Example 6.

In this Comparative Example, the differential pressure of the system rose to about 15 psid (pounds per square inch differential) near the end of the filtration indicating increased fouling of the functionalized nonwoven media in the capsule. In contrast, the step of loading the capsule with non-lysed AAV2 transfected cell culture resulted in a maximum differential pressure of only about 1 psid, indicating that greater loading of a filtration capsule can be achieved using the filtration procedure of Example 6 instead of the filtration procedure Comparative Example A.

### Example 8 (Ex. 8)

A reservoir containing AAV2 transfected cell culture was connected to the inlet port of a capsule prepared according to Ex. 1 using flexible tubing. A Masterflex L/S peristaltic pump was used to pump the cell culture fluid through the capsule at a constant flux of 200 LMH and throughput of 100 L/m². The cell culture in the reservoir was stirred throughout the procedure. After loading the capsule with the cell culture, a hypertonic aqueous NaCl solution (conductivity of 20 mS/cm) was pumped through the capsule at a constant flux of 200 LMH and throughput of 160 L/m² to elute AAV2 capsids from the functionalized nonwoven discs.

The resulting filtrate was collected and analyzed for AAV2 capsid content using a ProGen AAV2 Xpress ELISA kit. The turbidity of the culture before and after filtration with the filtration capsule was measured (using an ORION AQ4500 turbidity meter) to determine the percent reduction in turbidity that resulted from the filtration procedure. The turbidity of the cell culture before filtration through the filtration capsule was 540 NTU. The results are presented in Table 5.

### Example 9 (Ex. 9)

The same procedure as described in Example 8 was followed with the exception that the aqueous NaCl solution used for elution had a conductivity of 25 mS/cm. The AAV2 capsid content of the filtrate is presented in Table 5.

### Example 10 (Ex. 10)

The same procedure as described in Example 9 was followed with the exception that the AAV2 transfected cell culture was pumped through the capsule a throughput of 300 L/m² and at a constant flux of 200 LMH. The AAV2 capsid content of the filtrate is presented in Table 5.

### Comparative Example B (CEx. B). Alternative Detergent Lysis Procedure

TRITON-X100 detergent solution was added to a small sample (50 mL) of AAV2 transfected cell culture (Method C procedure) to achieve a final detergent concentration of 0.1% (volume/volume). To lyse the cells, the flask was placed on a shaker table in an incubator (set at 37° C., 8% CO₂) and shaken at 90 rpm for 2 hours. Following the incubation period, an aqueous sodium chloride solution (5 M) was added to adjust the conductivity of the cell culture to 20 mS/cm. The lysed cell culture fluid was centrifuged at 2500×g for one minute. The resulting supernatant was then filtered through a 0.2 micron PES (polyethersulfone) membrane filter. The filtrate was collected and analyzed for AAV2 capsid content using a ProGen AAV2 Xpress ELISA kit. The number of AAV2 capsids measured in the filtrate was used to calculate the corresponding number of capsids in a sample having the same volume as the culture sample loaded in Example 8. The result is presented in Table 5.

TABLE 5

| | Cell Culture | | Eluant | AAV2 | Reduction of |
|---|---|---|---|---|---|
| | Throughput | | Conductivity | Capsids in | Turbidity |
| Procedure | (L/m²) | Eluant | (mS/cm) | Filtrate | (%) |
| Ex. 8 | 100 | NaCl (5M) | 20 | 1.55 × 10¹¹ | 99.9% |
| Ex. 9 | 100 | NaCl (5M) | 25 | 1.76 × 10¹¹ | 99.2% |
| Ex. 10 | 300 | NaCl (5M) | 25 | 1.60 × 10¹¹ | 99.0% |
| CEx. B | N/A | N/A | N/A | 1.75 × 10¹¹ | ND |

N/A = not applicable
ND = not determined

### Example 11 (Ex. 11)

A reservoir containing AAV2 transfected cell culture was connected to the inlet port of a capsule prepared according to Ex. 1 using flexible tubing. A Masterflex L/S peristaltic pump was used to pump the cell culture fluid through the capsule at a constant flux of 200 LMH and throughput of 183 L/m². The cell culture in the reservoir was stirred throughout the procedure. After loading the capsule with the cell culture, a hypertonic phosphate-buffered saline solution (PBS) (1×, pH 7.4, conductivity: 15 mS/cm) was pumped through the capsule at a constant flux of 200 LMH to elute AAV2 capsids from the functionalized nonwoven discs (elution throughput: 183 L/m²). The resulting filtrate was collected and analyzed for AAV2 capsid content. The AAV2 capsid content of the filtrate was determined using a ProGen AAV2 Xpress ELISA kit. The results are presented in Table 6.

### Example 12 (Ex. 12)

The same procedure as described in Example 11 was followed with the exception that the conductivity of the PBS solution used to elute AAV2 capsids from the functionalized nonwoven discs was 21 mS/cm. The AAV2 capsid content of the filtrate is presented in Table 6.

### Example 13 (Ex. 13)

A reservoir containing AAV2 transfected cell culture was connected to the inlet port of a capsule prepared according to Ex. 1 using flexible tubing. A Masterflex L/S peristaltic pump was used to pump the cell culture fluid through the capsule at a constant flux of 200 LMH and throughput of 91 L/m². The cell culture in the reservoir was stirred throughout the procedure. After loading the capsule with the cell culture, a hypertonic 0.2 M sodium phosphate solution (conductivity: 20 mS/cm) was pumped through the capsule at a constant flux of 200 LMH to elute AAV2 capsids from the functionalized nonwoven discs (elution throughput: 91 L/m²). The resulting filtrate was collected and analyzed for AAV2 capsid content. The AAV2 capsid content of the filtrate was determined using a ProGen AAV2 Xpress ELISA kit. The results are presented in Table 6.

### Example 14 (Ex. 14)

The same procedure as described in Example 13 was followed with the exception that a 0.1 M sodium phosphate solution (conductivity: 11.5 mS/cm) was used to elute AAV2 capsids from the functionalized nonwoven discs. The AAV2 capsid content of the filtrate is presented in Table 6.

TABLE 6

| | Cell Culture | | Eluant | AAV2 |
|---|---|---|---|---|
| | Throughput | | Conductivity | Capsids in |
| Procedure | (L/m²) | Eluant | (mS/cm) | Filtrate |
| Ex. 11 | 183 | PBS | 15 | 5.75 × 10¹¹ |
| Ex. 12 | 183 | PBS | 21 | 1.7 × 10¹³ |
| Ex. 13 | 91 | sodium phosphate | 20 | 1.59 × 10¹³ |
| | | (0.2M) | | |
| Ex. 14 | 91 | sodium phosphate | 11.5 | 6.51 × 10¹² |
| | | (0.1M) | | |

### Example 15 (Ex. 15)

A reservoir containing AAV2 transfected cell culture was connected to the inlet port of a capsule prepared according to Ex. 1 using flexible tubing. A Masterflex L/S peristaltic pump was used to pump the cell culture fluid through the capsule at a constant flux of 200 LMH and throughput of 107 L/m². The cell culture in the reservoir was stirred throughout the procedure. After loading the capsule with the cell culture, a hypertonic solution of 50 mM Tris buffer (conductivity adjusted to 25 mS/cm using 5 M NaCl) was pumped through the capsule at a constant flux of 200 LMH and throughput of 100 L/m² to elute AAV2 capsids from the functionalized nonwoven discs. The resulting filtrate was collected and analyzed for AAV2 capsid content using a ProGen AAV2 Xpress ELISA kit. The results are presented in Table 7.

### Example 16 (Ex. 16)

The same procedure as described in Example 15 was followed with the exception that a filtration capsule prepared according to Example 2 was used. The cell culture fluid was pumped through the capsule at a constant flux of 200 LMH and throughput of 101 L/m². The Tris buffer was pumped through the capsule at a constant flux of 200 LMH and throughput of 100 L/m². The results are presented in Table 7.

### Example 17 (Ex. 17)

The same procedure as described in Example 15 was followed with the exception that a filtration capsule prepared according to Example 3 was used. The cell culture fluid was pumped through the capsule at a constant flux of 200 LMH and throughput of 169 L/m². The Tris buffer was pumped through the capsule at a constant flux of 200 LMH and throughput of 100 L/m². The results are presented in Table 7.

### Example 18 (Ex. 18)

The same procedure as described in Example 15 was followed with the exception that a filtration capsule prepared according to Example 4 was used. The cell culture fluid was pumped through the capsule at a constant flux of 200 LMH and throughput of 76 L/m². The Tris buffer was pumped through the capsule at a constant flux of 200 LMH and throughput of 100/m². The results are presented in Table 7.

### Example 19 (Ex. 19)

The same procedure as described in Example 15 was followed with the exception that a filtration capsule prepared according to Example 5 was used. The cell culture fluid was pumped through the capsule at a constant flux of 200 LMH and throughput of 125 L/m². The Tris buffer was pumped through the capsule at a constant flux of 200 LMH and throughput of 100 L/m². The results are presented in Table 7.

TABLE 7

| | Inlet to Outlet Orientation | Cell Culture | AAV2 |
|---|---|---|---|
| | of FNW Discs in | Throughput | Capsids in |
| Procedure | Filtration Capsule | (L/m²) | Filtrate |
| Ex. 15 | FNW-C/FNW-C/FNW-E/FNW-F | 107 | 1.95 × 10¹⁴ |
| Ex. 16 | FNW-C/FNW-D/FNW-E/FNW-F | 101 | 1.84 × 10¹³ |
| Ex. 17 | FNW-A/FNW-A/FNW-E/FNW-F | 169 | 2.18 × 10¹³ |
| Ex. 18 | FNW-C/FNW-C/FNW-C/FNW-E/FNW-F | 76 | 2.22 × 10¹³ |
| Ex. 19 | FNW-A/FNW-C/FNW-C/FNW-C/FNW-F | 125 | 1.77 × 10¹⁴ |

### Example 20 (Ex. 20)

A reservoir containing AAV2 transfected cell culture was connected to the inlet port of a capsule prepared according to Ex. 1 using flexible tubing. A Masterflex L/S peristaltic pump was used to pump the cell culture fluid through the capsule at a constant flux of 200 LMH and throughput of 183 L/m². The cell culture in the reservoir was stirred throughout the procedure. After loading the capsule with the cell culture, a hypertonic aqueous solution containing 1×PBS and 0.1 wt. % TRITON X-100 (conductivity: 15 mS/cm, pH 7.4) was pumped through the capsule at a constant flux of 200 LMH to elute AAV2 capsids from the functionalized nonwoven discs (elution throughput: 183 L/m²). The resulting filtrate was collected and analyzed for AAV2 capsid content. AAV2 capsid content of the filtrate was determined using a ProGen AAV2 Xpress ELISA kit. The results are presented in Table 8.

### Example 21 (Ex. 21)

The same procedure as described in Example 20 was followed with the exception that the conductivity of the solution used to elute AAV2 capsids from the functionalized nonwoven discs was 21 mS/cm. The AAV2 capsid content of the filtrate is presented in Table 8.

### Example 22 (Ex. 22)

A reservoir containing AAV2 transfected cell culture was connected to the inlet port of a capsule prepared according to Ex. 1 using flexible tubing. A Masterflex L/S peristaltic pump was used to pump the cell culture fluid through the capsule at a constant flux of 200 LMH and throughput of 91 L/m². The cell culture in the reservoir was stirred throughout the procedure. After loading the capsule with the cell culture, a hypertonic aqueous solution containing 0.2 M sodium phosphate and 0.1 wt. % TRITON X-100 (conductivity: 20 mS/cm) that was pumped through the capsule at a constant flux of 200 LMH to elute AAV2 capsids from the functionalized nonwoven discs (elution throughput: 91 L/m²). The resulting filtrate was collected and analyzed for AAV2 capsid content. AAV2 capsid content of the filtrate was determined using a ProGen AAV2 Xpress ELISA kit. The results are presented in Table 8.

### Example 23 (Ex. 23)

The same procedure as described in Example 22 was followed with the exception that an aqueous solution containing 0.1 M sodium phosphate and 0.1 wt. % TRITON X-100 (conductivity: 11.5 mS/cm) was used to elute AAV2 capsids from the functionalized nonwoven discs. The AAV2 capsid content of the filtrate is presented in Table 8.

TABLE 8

| | Cell Culture | | Eluant | AAV2 |
|---|---|---|---|---|
| | Throughput | | Conductivity | Capsids in |
| Procedure | (L/m²) | Eluant | (mS/cm) | Filtrate |
| Ex. 20 | 183 | PBS + 0.1 wt. % | 15 | 5.13 × 10¹¹ |
| | | TRTON X-100 | | |
| Ex. 21 | 183 | PBS + 0.1 wt. % | 21 | 1.24 × 10¹³ |
| | | TRITON X-100 | | |
| Ex. 22 | 91 | 0.2M sodium phosphate + | 20 | 2.13 × 10¹³ |
| | | 0.1 wt. % TRITON X-100 | | |
| Ex. 23 | 91 | 0.1M sodium phosphate + | 11.5 | 6.8 × 10¹² |
| | | 0.1 wt. % TRITON X-100 | | |

### Example 24 (Ex. 24)

A reservoir containing AAV2 transfected cell culture was connected to the inlet port of a capsule prepared according to Ex. 1 using flexible tubing. A Masterflex L/S peristaltic pump was used to pump the cell culture fluid through the capsule at a constant flux of 200 LMH and throughput of 100 L/m². The cell culture in the reservoir was stirred throughout the procedure.

After loading the capsule with the cell culture, hypotonic conditions were applied to the captured cells through the introduction of deionized water. Deionized water was pumped through the capsule at a constant flux of 200 LMH for a throughput of 62.5 L/m². Following application of the deionized water, the pump was shut off and the captured cells were allowed to equilibrate and lyse with the water held up in the capsule for approximately 30 minutes. Following the hypotonic equilibration, a hypertonic solution of Tris buffer (50 mM, conductivity of 25 mS/cm) was pumped through the capsule at a constant flux of 200 LMH to elute AAV2 capsids from the functionalized nonwoven discs (elution throughput: 100 L/m²). The resulting filtrate was collected and analyzed for AAV2 capsid content using a ProGen AAV2 Xpress ELISA kit. The lysis process resulted in the recovery of 2.42×10¹¹ AAV2 capsids.

All references and publications cited herein are expressly incorporated herein by reference in their entirety into this disclosure. Illustrative embodiments of this invention are discussed and reference has been made to possible variations within the scope of this invention. For example, features depicted in connection with one illustrative embodiment may be used in connection with other embodiments of the invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A method, the method comprising:
providing a filter medium comprising a functionalized nonwoven;
passing a first fluid containing cells through the filter medium, wherein at least some of the cells are captured by the functionalized nonwoven;
passing a second fluid, and optionally a third and/or fourth fluid through the filter medium, wherein the second and/or third and/or fourth fluid disrupts at least one of the captured cells; and
recovering an intracellular biological product in the first and/or second and/or third and/or fourth fluid.

2. The method of claim 1, wherein the second fluid has an osmotic potential less than the osmotic potential of the first fluid, and wherein the change in osmotic potential within the filter medium results in a disruption of at least one of the cells.

3. The method of claim 2, wherein the second fluid is a sucrose solution or a salt solution.

4. The method of claim 1, wherein the second fluid has an osmotic potential more than the osmotic potential of the first fluid, and wherein the change in osmotic potential within the filter medium results in a disruption of at least one of the cells.

5. The method of claim 4, wherein a conductivity of the second fluid is less than a conductivity of the first fluid.

6. The method of claim 4, wherein the second fluid is water.

7. The method of claim 1, wherein the second fluid comprises a surfactant or a chemical cell lysis agent.

8. The method of claim 7, wherein the surfactant is selected from the group consisting of non-ionic surfactants, cationic surfactants, a net charge of zero (zwitterionic detergents), and mixtures thereof.

9. The method of claim 1, wherein a conductivity of the third fluid is different than that of the first fluid and different than that of the second fluid.

10. The method of claim 2, wherein a conductivity of the second fluid is greater than a conductivity of the first fluid.

11. The method of claim 9, wherein the third fluid is water.

12. The method of claim 1, wherein the third or fourth fluid is a conductive salt solution with a conductivity ranging from 9 to 60 mS/cm.

13. The method of claim 12, wherein the third or fourth fluid is selected from NaCl solution, Phosphate Buffered Saline, Phosphate Buffer, Tris-HCl buffer, Tris-Acetate buffer, HEPES Buffer [4-(2-hydroxyethyl)-1-piperazine ethane sulfonic acid].

14. The method of claim 1, wherein the functionalized nonwoven has a dynamic charge capacity.

15. The method of claim 1, wherein the functionalized nonwoven is cationically charged.

16. The method of claim 1, wherein at least some of the cells are captured electrostatically by the functionalized nonwoven.

17. The method of any of claim 1, wherein the functionalized nonwoven is grafted with copolymers comprising interpolymerized monomer units of a quaternary ammonium containing monomer, an amide containing monomer, and an epoxy containing monomer.

18. The method of claim 17, wherein the monomer is MAPTAC (Methacrylamidopropyltrimethyl-ammonium chloride monomer

19. The method of any claim 1, wherein the filter medium comprises multiple layers of functionalized nonwoven, each layer having a same or different calculated pore size and a same or different dynamic charge capacity.

20. The method of any of claim 1, wherein intracellular biological product comprises AAV capsids, therapeutic/recombinant proteins, plasmids, DNA, RNA, viruses, virus like particles, exosomes, and mixtures thereof.

21. The method of any claim 1, wherein at least some of nucleic acids are captured by the functionalized nonwoven.

22. The method of any of claim 1, wherein the functionalized nonwoven reduces DNA concentrations in the first fluid below 10 ng/ml.
